(19) United States
(12) Patent Application Publication
(10) Pub. No.: US 2004/0186303 A1
(43) Pub. Date: Sep. 23, 2004

(54) Burning an exhaust gas containing oxygen and a combustible component

(76) Inventor: Walter Schicketanz, Frankenthal (DE)

Correspondence Address: Herbert B. Keil, KEIL & WEINKAUF, 1350 Connecticut Ave., N.W., Washington, DC, 20036, US

(21) Appl. No.: 10/765,987

(22) Filed: Jan. 29, 2004

(30) Foreign Application Priority Data
Feb. 3, 2003 (DE) 10304154.0

(51) Int. Cl. C07D 307/89; C07D 307/36; C07C 51/16

(52) U.S. Cl. 549/248; 562/545; 549/262

(57) ABSTRACT

A process for burning in a combustion chamber an exhaust gas containing oxygen and a combustible component, which exhaust gas originates from the heterogeneously catalyzed gas-phase oxidation of an inorganic or organic compound, by heating the exhaust gas to a temperature in the range from 200° C. to a temperature which corresponds to the hottest temperature in the heterogeneously catalyzed gas-phase oxidation and is above 200° C. and feeding the exhaust gas at this temperature to the burner head.

The present invention relates to a process for burning an exhaust gas containing oxygen and a combustible component in a combustion chamber, which exhaust gas originates from the heterogeneously catalyzed gas-phase oxidation of an inorganic or organic compound.

Exhaust gases which contain a combustible component together with oxygen are formed in many different heterogeneously catalyzed oxidation processes. Owing to the simultaneous presence of an oxidizing agent (oxygen) and a combustible component, thermal purification of such exhaust gases, that is to say their combustion, requires special safety measures, in particular with respect to safely and reliably avoiding flashbacks.

An overview of oxidative processes for purifying exhaust gases, in particular of catalytic and thermal purification processes, may be found in J. M. Klobucar, Chem. Eng., February 2002, pages 62 to 67.

In catalytic exhaust gas purification, the exhaust gas is catalytically converted into more environmentally friendly compounds at temperatures of typically from 200 to 650° C. in the presence of air and a catalyst. The use of catalysts makes far lower operating temperatures possible compared with pure combustion of the exhaust gas, which leads to advantages in the overall energy balance and choice of materials. The disadvantages of catalytic exhaust gas purification are closely connected to the use of catalysts. These usually contain noble metals, for example palladium or platinum, and therefore have a tendency, on contact with various compounds, to reversible or irreversible damage. If such compounds, termed catalyst poisons, are expected, generally a guard bed is provided upstream. Since the catalysts have only a limited service life, even in the absence of catalyst poisons, for reliable and long-lasting operation of a catalytic exhaust gas purification process, frequently the oxidation reactor must be constructed in duplicate. Furthermore, in the case of exhaust gases having a high content of combustible components, there is the risk of excessively high reaction temperatures and flashback, and also the risk of damage to the catalyst and to the plant.

In the thermal purification of exhaust gas, the exhaust gas is burnt at temperatures of typically from 800 to 1000° C. in the presence of air with or without what is called a supplemental fuel to form more environmentally friendly compounds, generally water and carbon dioxide. Generally, a differentiation is made between a direct flame oxidizer, a recuperative oxidizer and a regenerative oxidizer.

In the case of the direct flame oxidizer, the non-preheated exhaust gas to be purified is burnt with air in a flame which is generated by a supplemental fuel, for example natural gas or oil. To avoid flashback, generally at the inlet to the combustion chamber, there is a high-velocity path in which the flow velocity of the exhaust gas fed is higher than the flashback velocity. A disadvantage of the direct flame oxidizer is the high consumption of supplemental fuel, in particular at low concentration of combustible components, since the exhaust gas to be burnt is fed in relatively cold and thus must be first brought to the desired combustion temperature with the aid of the heat of combustion of the supplemental fuel.

In the recuperative oxidizer, the non-preheated exhaust gas to be purified is preheated by the waste heat of the ideally autothermal combustion in the oxidizer and then burnt with air in the actual combustion chamber. The preheating generally takes place in such a manner that the exhaust gas fed, before entry into the combustion chamber, first flows through a heat exchanger which is operated on the other side with the hot flue gas. If the content of combustible components is not sufficient for autothermal combustion, the missing energy can be introduced by an auxiliary burner. Only by said preheating is substantially autothermal combustion made possible, since the exhaust gas to be burnt already flows hot into the combustion chamber. However, precisely this also has a critical disadvantage. Since with increasing temperature in the exhaust gas its readiness to ignite also increases, there is the risk of flashback into the heat exchanger and thus the danger of relatively severe damage. This danger is the more distinct, the higher the concentration of combustible components and the lower the flow velocity. Therefore, in particular in the case of exhaust gases having a high concentration of combustible components and/or great fluctuations in composition and rate, the safe use of a recuperative oxidizer is not ensured.

In the regenerative oxidizer, the non-preheated exhaust gas to be purified is preheated via a hot heat storage medium and is burnt autothermally under ideal conditions in a downstream combustion chamber. The hot flue gases are then passed over a second heat storage medium which at the time is in the regenerative mode, and heat it up. If the first-mentioned heat storage medium has fallen in temperature to the extent that the desired combustion is no longer ensured, the flow is crossed over and the second heated heat storage medium is used for heating up. If the content of combustible components is not sufficient for the autothermal combustion, the missing energy can be introduced via an auxiliary burner. In the regenerative oxidizer also, substantially autothermal combustion is only made possible by said preheating. As already described above in the case of the recuperative oxidizer, in the case of the regenerative oxidizer there is also the danger with exhaust gases of high concentration of combustible components that the oxidation reaction will run away as soon as in the bed of the heat storage medium, that is to say will lead to an uncontrolled temperature increase which can damage the plant. There is also the danger of flashback into the heat exchanger and thus the danger of relatively severe damage. Therefore, in the case of exhaust gases with a high concentration of combustible components and/or great fluctuations in the composition and rate, the safe use of a regenerative oxidizer is not ensured. In addition, the regenerative oxidizer, owing to its at least two heat storage chambers, each of which is designed to heat up the non-preheated exhaust gas, is very large and costly in terms of apparatus.

To prevent flashback safely, in the exhaust gas feed to the oxidizer, generally, depending on the hazard potential, a plurality of safety measures, which are independent of each other, are used, such as flame barriers and/or dilution of the exhaust gas and/or analytical instruments which are installed upstream and analyze the ignition behavior of the exhaust gas. Overviews of this may be found, for example, in G.-G. Börger et al., VDI-Berichte No. 286,1977, pages 131 to 134, in K. Schampel et al., Gas wärme international 27, 1978, November, pages 629 to 635, and in W. Hüning, Chem.-Ing.-Tech. 57,1985, pages 850 to 857. Known flame barriers are, for example, liquid seals, flame arresters, screens, detonation arresters, high-velocity pathways, feeds of fresh air or flashback-proof nozzle feeds into the combustion chamber. The exhaust gas can be appropriately diluted, for example, with air. Thus, in the last-mentioned literature reference, in FIG. 5 there, a combination of liquid seal, fresh air feed with high-velocity path, detonation arrester and flashback-proof nozzle feed into the combustion chamber [lacuna]. Although the use of a high-velocity path, when the required minimum velocity is maintained, does make reliable prevention of flashback possible, it has the critical disadvantage that by feeding further air to maintain the required flow velocity, the total amount of exhaust gas increases and thus in some cases also, the energy requirement for heating it up prior to combustion increases.

It is an object of the present invention, therefore, to find a process for purifying an exhaust gas containing oxygen and a combustible component, which does not have the abovementioned disadvantages, ensures safe long-term operation, is substantially autonomous from the energy point of view even when the exhaust gas produced markedly falls below the lower explosive limit and, in particular, also copes with changing exhaust gas rates and changing exhaust gas compositions without any problem.

We have found that this object is achieved by a process for burning in a combustion chamber an exhaust gas containing oxygen and a combustible component, which exhaust gas originates from the heterogeneously catalyzed gas-phase oxidation of an inorganic or organic compound, which comprises heating the exhaust gas to a temperature in the range from 200° C. to a temperature which corresponds to the hottest temperature in the heterogeneously catalyzed gas-phase oxidation and is above 200° C., and feeding the exhaust gas at this temperature to the burner head.

For the purposes of the present invention combustion means the thermal reaction with oxygen of the combustible component present in the exhaust gas, which reaction customarily takes place in a temperature range from 700 to 1200° C. The combustion takes place in a combustion chamber into which the exhaust gas to be burnt is introduced. At an appropriately high content of combustible components and/or an appropriately high temperature of the exhaust gas fed, autothermal combustion may be possible. Autothermal combustion is distinguished by the required fuel originating solely from the exhaust gas to be burnt. If the content of combustible components and/or the temperature of the exhaust gas is correspondingly low, the use of an auxiliary or supplemental burner can be necessary. This may be a separate burner or be integrated into the abovementioned burner head and is operated with an additional fuel, for example natural gas or oil, and supplies the remaining energy required for the combustion. Generally, combustion chambers which are operated autothermally, also contain a supplementary burner in order, in particular, to make it possible to start up the plant and, in the event of fluctuations or disruptions to the exhaust gas feed, to ensure continuous combustion.

In the combustion the exhaust gas fed is converted into predominantly more environmentally friendly compounds. If the exhaust gas contains, as combustible components, only hydrogen-, carbon- and/or oxygen-containing compounds, these are generally reacted to form water and carbon dioxide. If the exhaust gas, in addition, contains further elements, for example chlorine or sulfur, these are converted into more stable compounds of chlorine or sulfur, for example hydrogen chloride, chlorine oxides or sulfur oxides. The gas obtained by the oxidative reaction is termed flue gas.

The exhaust gas to be used in the inventive process originates from the heterogeneously catalyzed gas-phase oxidation of an inorganic or organic compound. Heterogeneously catalyzed gas-phase reactions are generally known to those skilled in the art. In heterogeneously catalyzed gas-phase oxidation, the starting material to be oxidized is passed in the gaseous state, together with a gas containing oxygen, through a suitable reactor which contains a heterogeneous catalyst and is oxidized at an elevated temperature in the range from customarily 200 to 600° C. to the desired product of value and by-products. Because the oxidation reactions are generally highly exothermic, generally salt-bath-cooled shell-and-tube reactors are used for this. The reaction gas passed out of the reactor thus contains the desired product of value, possible by-products, unreacted starting material, the gaseous water of reaction formed in the reaction and remaining unreacted oxygen. Generally the reaction gas passed out of the reactor is cooled and the product of value separated off. The product of value can be separated off in many ways. Suitable possible methods are, for example, absorption in a solvent, condensation or desublimation. Depending on the embodiment and type of the heterogeneously catalyzed gas-phase oxidation process, following the separation of the product of value, further steps can also follow, for example, for removing or reducing the water of reaction, for washing, for extractions or for distillations. In addition, it is of course also possible to recirculate to the reactor a portion of the gas which still contains residual unreacted starting material (recycle mode). The remaining gas to be disposed of by combustion is termed exhaust gas. It is emphasized that, in the context of the present invention, it is not critical from which heterogeneously catalyzed gas-phase reaction the exhaust gas to be burnt originates, provided that it contains oxygen and a combustible component.

It is essential in the inventive process that the exhaust gas is heated to a temperature in the range from 200° C. to a temperature which corresponds to the hottest temperature in the heterogeneously catalyzed gas-phase oxidation and is above 200° C., and is fed at this temperature to the burner head. The burner head is a piece of apparatus which serves for feeding the exhaust gas to the combustion chamber and to form the flame. Generally the burner head has measures for gas distribution and vortexing, flame retention and if appropriate an integrated ignition mechanism, and also a flame detector. Preferably, in the inventive process, flashback-proof burner heads are used, as are described, for example, in G.-G. Börger et al., VDI-Berichte No. 286, 1977, page 133, FIG. 5 and associated text.

Said lower limit of the temperature range also makes possible the autothermal combustion of exhaust gases having a low content of combustible components, since owing to the preheating in the region of the flame, only a relatively small amount of heat is in that case required for heating up to the ignition temperature. Without corresponding preheating, in the case of exhaust gases having a low content of combustible components, the heat liberated in the flame under some circumstances could no longer suffice to heat the exhaust gas up to the ignition temperature, which would lead to the flame extinguishing. In practice, this would then mean the use of supplemental fuel.

In principle, it holds that with increasing exhaust gas temperature, exhaust gases having a decreasing content of combustible components can also be burnt autothermally. Preference is therefore given to a process in which the exhaust gas is heated to a temperature in the range from 300° C. to a temperature which corresponds to the hottest temperature in the heterogeneously catalyzed gas-phase oxidation and is above 300° C., and is fed at this temperature to the burner head.

Particular preference is given to a process in which the exhaust gas is heated to a temperature in the range from 50° C. below the temperature corresponding to the hottest temperature in the heterogeneously catalyzed gas-phase oxidation to a temperature corresponding to the hottest temperature in the heterogeneously catalyzed gas-phase oxidation and is fed at this temperature to the burner head.

Said upper limit of the temperature range ensures that the exhaust gas is always in a temperature range in which an explosion without additional ignition source, and thus an explosion in the exhaust gas system, is ruled out. The effect is ultimately based on the fact that in the heterogeneously catalyzed gas-phase oxidation, an appropriate temperature was already present in the reactor, and the reaction mixture, owing to the oxidation reaction and the subsequent separation of the product of value, is depleted in combustible components, and also these, compared with the components at the point of the hottest temperature in the heterogeneously catalyzed gas-phase oxidation, are lower in energy owing to the higher degree of oxidation, and the lower explosive limit thereof is thus even higher. In this context the lower explosive limit is the defining explosive limit under the existing pressure and existing gas composition.

For the sake of completeness, it may be mentioned that the hottest temperature in the heterogeneously catalyzed gas-phase oxidation is generally also called the hot spot temperature.

For safety reasons, the heterogeneously catalyzed gas-phase oxidation of the inorganic or organic compound from which the exhaust gas to be burnt originates is preferably carried out in a region below the lower explosive limit. This means that at all points in the heterogeneously catalyzed gas-phase oxidation process, at the existing temperature, the existing pressure and the existing gas composition, conditions fall below the lower explosive limit.

The temperature of the exhaust gas originating from the heterogeneously catalyzed gas-phase oxidation process is generally below the hottest temperature of the heterogeneously catalyzed gas-phase oxidation and generally also below 200° C. Therefore, in the inventive process, the exhaust gas is generally preheated to the desired temperature. The preheating can be performed directly or indirectly. In the case of direct preheating, hot gas, preferably hot flue gas, is admixed under temperature control to the exhaust gas. In the case of indirect preheating, the exhaust gas is heated via a heat exchanger. This can be operated, for example, by the hot flue gas, the hot salt melt from the reactor of the heterogeneously catalyzed gas-phase oxidation, or by another heat source, for example superheated steam. Preferably, the exhaust gas is heated via a heat exchanger which is heated by the flue gas being released by the combustion. This enables energetically autonomous heating of the exhaust gas.

Heating the exhaust gas via a heat exchanger which is heated by the flue gas released by the combustion can be implemented in many ways. For instance, it is possible, for example, to control the temperature in the exhaust gas via the ratio between the exhaust gas stream flowing through the heat exchanger and an exhaust gas stream flowing through a bypass. In this variant, therefore, a portion of the exhaust gas stream is passed, to preheat it, through a heat exchanger operated by the flue gas, whereas the other portion of the flue gas is passed through a bypass around the heat exchanger. The two streams are then recombined. Generally, the mixture temperature is measured continuously and the exhaust gas ratio between heat exchanger and bypass is controlled by a comparison with the desired preset temperature. If the mixture temperature is above the preset temperature, for instance, in the simplest case the mixture temperature is adjusted downward by increasing the exhaust gas stream which is passed through the bypass and decreasing the exhaust gas stream which is passed through the heat exchanger, and vice versa. Advantageously, the temperature in the exhaust gas stream which leaves the heat exchanger is also measured continuously and is kept, via a further control circuit, at a temperature which corresponds at the maximum to the hottest temperature in the heterogeneously catalyzed gas-phase oxidation. Possible measures for ensuring that said maximum temperature is not exceeded are, for example, feeding cold and preferably low-oxygen gas upstream of the heat exchanger or controlling the rate or the temperature of the flue gas flowing through the heat exchanger. The rate of the flue gas flowing through can be controlled, for example, by a control flap valve in the flue gas system upstream of the heat exchanger and corresponding bypass for the remaining flue gas volume around the heat exchanger. The temperature of the flue gas flowing through can result from, for example, mixing flue gases of differing temperatures by partial and controlled recycling of colder flue gas which is present downstream, for example, after being passed through further heat exchangers.

Preferably, in the inventive process, the temperature in the exhaust gas is controlled via the ratio between the exhaust gas stream flowing through the heat exchanger and an exhaust stream flowing through a bypass and, in addition, the temperature at the outlet of the heat exchanger via the volumetric flow rate of the flow gas flowing through the heat exchanger. Said volumetric flow rate can be controlled, as described above, for example by a control flap valve in the flue gas system upstream of the heat exchanger and corresponding bypass for the remaining flue gas volume.

Despite the abovementioned measures, in order to actually rule out flashback, for further safety, in the inventive process, in the exhaust gas feed, generally one or more further safety measures against flashback are used. Suitable measures are high-velocity paths, high-velocity valves, flashback preventers, such as liquid seals, flame arresters, screens, detonation safeguards and measures such as flashback-free nozzle feed into the combustion chamber. They are described, for example, in G.-G. Börger et al., VDI-Berichte No. 286,1977, pages 131 to 134, in K. Schampel et al., Gas wärme international 27,1978, November, pages 629 to 635 and in W. Hüning, Chem.-Ing.-Tech. 57,1985, pages 850 to 857. Depending on the type of exhaust gas and the safety desired, a plurality of these safety measures can also be used in series. Preferably, upstream of the inlet into the combustion chamber is situated a flashback preventer, which, in particular, prevents flashback in the event of a sudden fault in operation. Furthermore, it may in some cases be advantageous to pass the heated exhaust gas, before it is introduced into the combustion chamber, through a high-velocity path or high-velocity valve, the flow velocity of the gas flowing through preferably being higher than the flashback velocity. In a particularly preferred embodiment using a high-velocity path, the required high gas velocity can be achieved by partial recirculation of flue gas.

In a particularly preferred variant of the inventive process, the hot flue gas formed is utilized energetically not only to preheat the exhaust gas, as described above, but also to heat up external energy carriers. Energetic utilization means here, in particular, production of hot water, steam and superheated steam. The corresponding processes for the energetic utilization of the flue gas and the apparatuses required therefor are generally known to those skilled in the art.

Combustible components in the exhaust gas which come into consideration in the inventive process are in principle all inorganic or organic compounds which are oxidizable by oxygen and which are gaseous under the existing conditions and originate from the heterogeneously catalyzed gas-phase oxidation of an inorganic or organic compound. The combustible component can be a single compound or a mixture of different compounds. Suitable combustible components are, for example, hydrogen, aliphatic, aromatic or araliphatic hydrocarbons, alcohols, aldehydes, ketones, carboxylic acids, ammonia or amines. Generally, the exhaust gas to be disposed of contains from 0.01 to 10% by volume, preferably from 0.01 to 5% by volume, and particularly preferably from 0.1 to 2% by volume, of combustible components.

Preferably, in the inventive process, an exhaust gas is used which originate from the heterogeneously catalyzed gas-phase oxidation of n-butane and/or n-butenes to maleic anhydride, of o-xylene to phthalic anhydride, of propene to acrylic acid, of isobutene to methacrylic acid, of 1,2-ethanediol to glyoxal, of ethene to ethylene oxide, of propene to acrolein, of propene and ammonia to acrylonitrile, of olefins to aldehydes or ketones, of methanol to formaldehyde and/or of methane and ammonia to hydrocyanic acid, and particularly preferably of n-butane and/or n-butenes to maleic anhydride, of o-xylene to phthalic anhydride, of propene to acrylic acid, of isobutene to methacrylic acid, of 1,2-ethanediol to glyoxal and/or of ethene to ethylene oxide.

Some preferred embodiments are described in more detail below with reference to simplified process flow diagrams. The apparatuses and valves are given capital letters and are named in the description. The lines are numbered consecutively in arabic numerals. The inputs and outputs of material streams are numbered in roman numerals and are likewise described in more detail in the description. The control and instrumentation equipment carries the conventional nomenclature having a number suffix for consecutive numbering, where “T” is temperature measurement and “C” is control circuit.

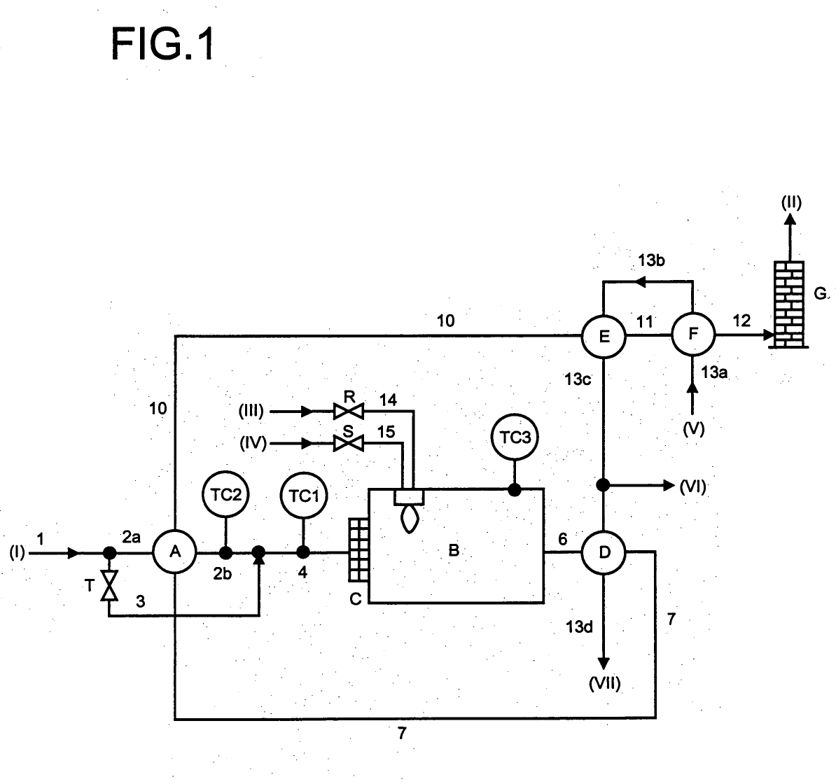

The simplified process flow diagram of a preferred embodiment having an exhaust-gas-side bypass round the flue gas/exhaust gas heat exchanger is shown in FIG. 1. The exhaust gas (I) originating from the heterogenously catalyzed gas-phase oxidation is supplied via line (1). A substream of the exhaust gas is conducted, for preheating, via line (2a) and line (2b) through a flue gas-operated heat exchanger (A). The other substream of the exhaust gas is bypassed round the heat exchanger via the bypass valve (T) and line (3) and combined within line (4) with the preheated exhaust gas. This is then passed via a static flashback preventer (C), preferably a screen, and fed to the combustion chamber (B). This comprises one or more burner heads (not shown) and contains an auxiliary burner, in particular for start-up and also for using exhaust gases which cannot be burned autothermally, which auxiliary burner can be present as a separate burner or integrated in the abovementioned burner head or burner heads and which, as required, can be operated with air (III) via line (14) and with fuel (IV), for example natural gas, via line (15). In the combustion chamber, in which further burners for other substances and exhaust gases can be integrated, the exhaust gas is oxidatively converted to the flue gas. This leaves the combustion chamber (B) in a hot state and is conducted via line (6) to a number of series-connected heat exchangers. In heat exchanger (D), superheated steam (VII) is produced. The flue gas is then conducted via line (7) through the flue gas/exhaust gas heat exchanger (A) and passed on via line (10), and in heat exchanger (E) saturated steam, and in heat exchanger (F) hot water (feed water preheating) are generated. The energetically utilized and cooled flue gas passes via line (12) to the stack (G) and is released as flue gas (II) into the atmosphere. At this point it may be noted that in the case of a flue gas containing pollutants, for example sulfur oxides or chlorine compounds, before emission into the atmosphere, various purification apparatuses can further be connected intermediately. The energy of the hot flue gas is utilized as shown by way of example in FIG. 1 by producing steam (VI) and/or superheated steam (VII) using the abovementioned heat exchangers (F), (E) and (D). It may be mentioned that, depending on embodiment, the heat exchanger (D) can also be integrated, for example, in the combustion chamber, so that line (6) would effectively disappear.

The control circuits of the preferred embodiment are described in more detail below. For better clarity, the control lines have been omitted in FIG. 1. “TC1” measures the exhaust gas temperature after combination of the exhaust gas preheated in heat ex-changer (A) and the exhaust gas bypassing the heat exchanger (A). This measured value serves for temperature control of the preheated exhaust gas and, in accordance with the preset value, controls, via the valve (T), the ratio between the exhaust gas flowing through the heat exchanger (A) and the exhaust gas bypassing the heat exchanger (A). “TC2” measures the temperature of the exhaust gas preheated in the heat exchanger (A) and, when the set maximum value, which is generally orientated on the hottest temperature in the heterogeneously catalyzed gas-phase oxidation, is achieved, it triggers measures which are to prevent exceedance of this maximum value. One of the suitable measures which is mentioned by way of example is controlled feed of cold gas, for example an inert flushing gas (for the sake of clarity, not designated in FIG. 1). If the temperature in the combustion chamber (B) reaches the upper limit of the desired temperature range, to hold the temperature, the feed of additional (ambient) air into the combustion chamber can be activated via “TC3”. This can be fed, for example, via valve (R) and line (14) or via an additional apparatus which is not shown in FIG. 1. Furthermore, it is also possible to activate the bypass valve (T) also via “TC3′, in order to lower the temperature of the exhaust gas introduced into the combustion chamber. This comes into consideration, in particular, when the exhaust gas has a high content of combustible components, and thus its energy content is relatively high. Furthermore, it may be advantageous in some cases to supplement said controls by further safety measures, for example by monitoring the wall temperature of the heat exchanger (A), by analytical instruments installed upstream which analyze the readiness to ignite of the exhaust gas downstream of the reactor or after separating off the product of value and if appropriate also trigger safety measures in the region of the reactor operation.

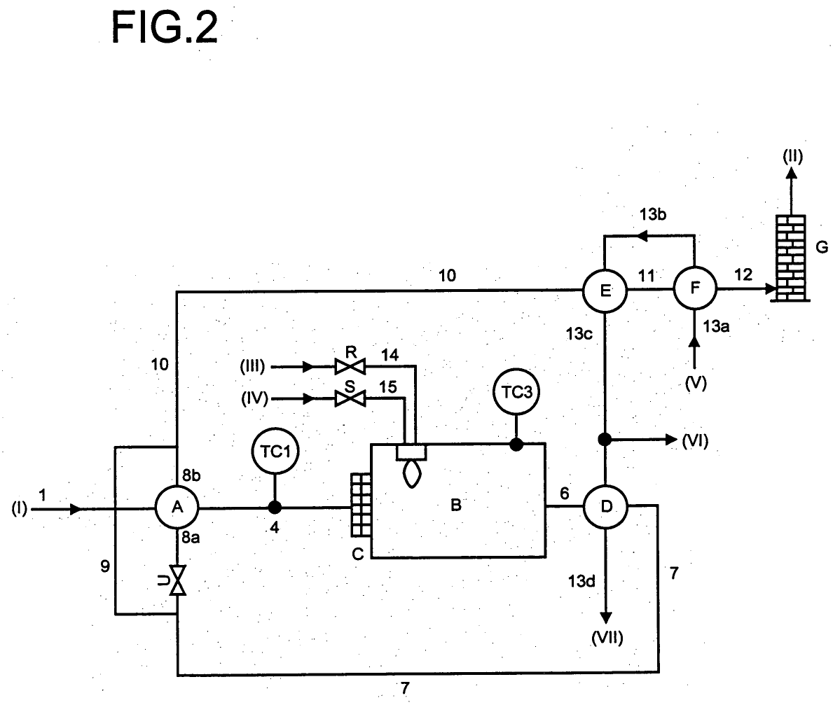

FIG. 2 shows the simplified process flow diagram of a preferred embodiment having a flue-gas-side bypass round the flue gas/exhaust gas heat exchanger. In contrast to the process shown in FIG. 1, in the present embodiment, the exhaust gas temperature is controlled via flue-gas-side control of the heat exchanger (A) and all of the exhaust gas is passed through the heat exchanger (A). In the flue-gas-side control, a substream of the flue gas is conducted through the heat exchanger (A) via the flue gas flap valve (U), line (8a) and (8b). The other flue gas substream is passed round the heat exchanger via the bypass line (9). In this embodiment “TC1” controls the flue gas flap valve (U) in accordance with the preset value and thus the flue gas rate flowing through the heat exchanger (A). In addition to the measures mentioned in the above-mentioned embodiment, “TC3” can also activate the flue gas flap valve (U), in order, as required, for example, to lower the temperature of the exhaust gas introduced into the combustion chamber.

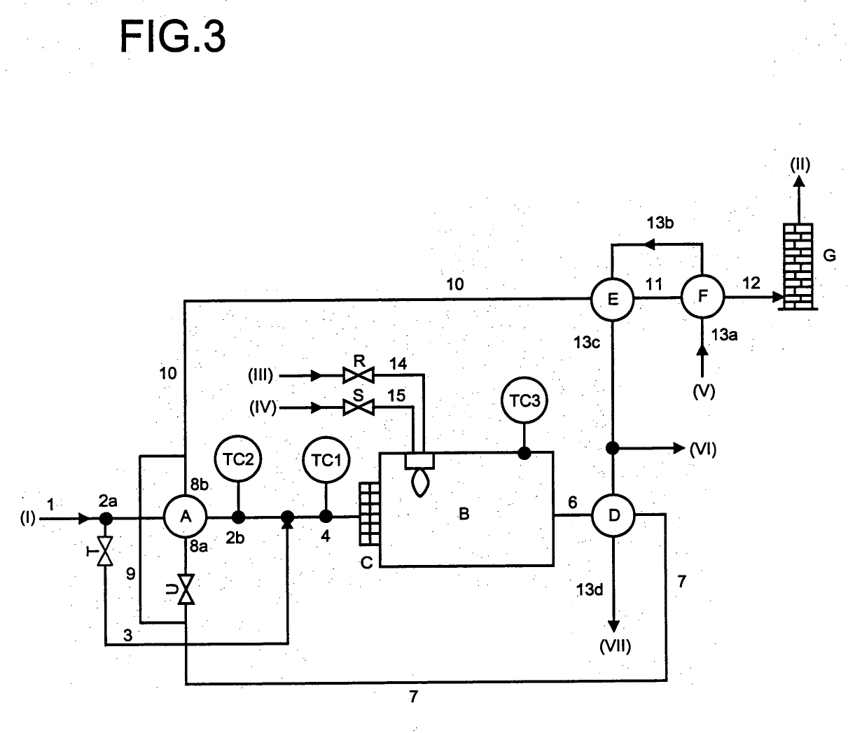

FIG. 3 shows the simplified process flow diagram of a preferred embodiment having exhaust-gas- and flue-gas-side bypass round the flue gas/exhaust gas heat exchanger. In this embodiment, the desired temperature of the exhaust gas preheated in the heat exchanger (A) is set by means of a flue-gas-side control via “TC2” using the flue-gas flap valve (U). The mixture temperature of the exhaust gas which is fed to the combustion chamber is set via “TC1” by means of the bypass valve (T). In addition to the measures mentioned in the first-mentioned embodiment, “TC3” can, as required, actuate not only the bypass valve (T) but also the flue gas flap valve (U) in order, as required, for example, to lower the temperature of the exhaust gas introduced into the combustion chamber.

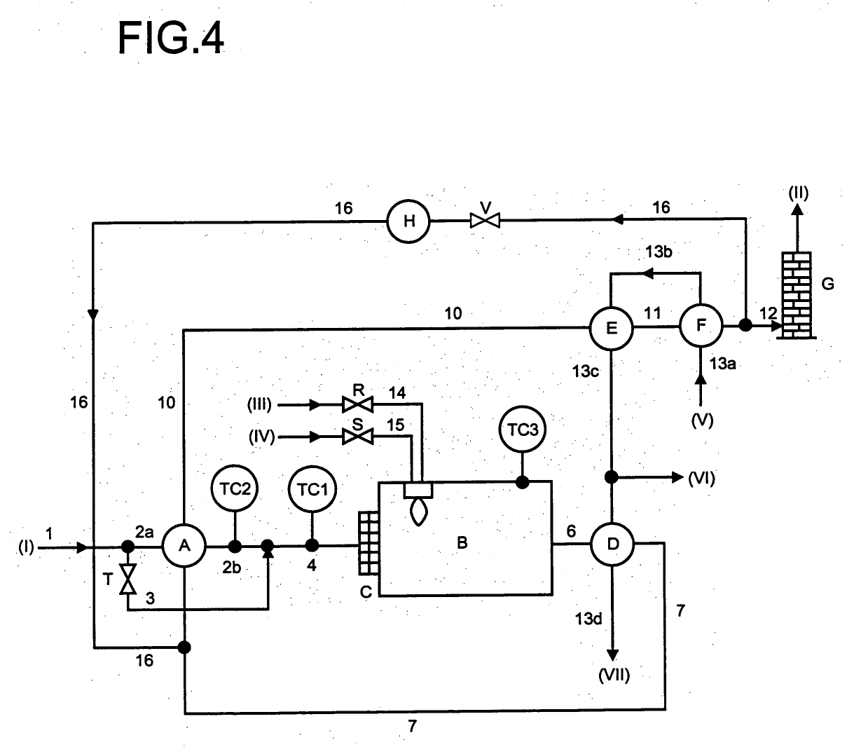

FIG. 4 finally shows the simplified process flow diagram of a preferred embodiment having exhaust-gas-side bypass round the flue gas/exhaust gas heat exchanger and partial recirculation of colder flue gas by means of a fan (H). In this embodiment, for example, when the preset maximum exhaust gas temperature is reached downstream of the heat exchanger (A), the recycle valve (V) can be actuated via “TC2”, in order to recirculate colder flue gas to the heat exchanger via line (16). In addition to the measures mentioned in the first-mentioned embodiment, “TC3” can, as required, actuate not only the bypass valve (T), but also the recycle valve (V), in order, as required, for example, to lower the temperature of the exhaust gas introduced into the combustion chamber.

The inventive process enables the combustion, in a combustion chamber, of an exhaust gas containing oxygen and a combustible component, which exhaust gas originates from the heterogeneously catalyzed gas-phase oxidation of an inorganic or organic compound, ensuring safe long-term operation. The inventive process, from the energy aspect, is substantially autonomous even with conditions markedly falling below the lower explosive limit of the exhaust gas produced and copes, in particular, even with changing exhaust gas rates and changing exhaust gas compositions without any problem. By heating the exhaust gas to a temperature which corresponds at least to said lower limit of the temperature range, autothermal combustion is promoted, even with an exhaust gas having a low content of combustible components, since owing to the preheating in the region of the flame, only a relatively small amount of heat is required for heating it up to the ignition temperature. Said upper limit of the temperature range ensures that the exhaust gas is always in a temperature range in which an explosion without additional ignition source, and thus an explosion in the exhaust gas system, is ruled out. The invention process, furthermore, is relatively simple to implement and to operate

We claim:

1. A process for burning in a combustion chamber an exhaust gas containing oxygen and a combustible component, which exhaust gas originates from the heterogeneously catalyzed gas-phase oxidation of an inorganic or organic compound, which comprises heating the exhaust gas to a temperature in the range from 200° C. to a temperature which corresponds to the hottest temperature in the heterogeneously catalyzed gas-phase oxidation and is above 200° C., and feeding the exhaust gas at this temperature to the burner head.

2. A process as claimed in claim 1, wherein the exhaust gas is heated to a temperature in the range from 300° C. to a temperature which corresponds to the hottest temperature in the heterogeneously catalyzed gas-phase oxidation and is above 300° C., and the exhaust gas is fed at this temperature to the burner head.

3. A process as claimed in claim 1, wherein the exhaust gas is heated to a temperature in the range from 50° C. below the temperature corresponding to the hottest temperature in the heterogeneously catalyzed gas-phase oxidation to a temperature corresponding to the hottest temperature in the heterogeneously catalyzed gas-phase oxidation and the exhaust gas is fed at this temperature to the burner head.

4. A process as claimed in claim 1, wherein the heterogeneously catalyzed gas-phase oxidation of the inorganic or organic compound is carried out in a range below the lower explosive limit.

5. A process as claimed in claim 1, wherein the exhaust gas is heated via a heat exchanger which is heated by the flue gas being liberated by the combustion.

6. A process as claimed in claim 5, wherein the temperature in the exhaust gas is controlled via the ratio between the exhaust gas stream flowing through the heat exchanger and an exhaust gas stream flowing through a bypass.

7. A process as claimed in either of claim 5 or 6, wherein the temperature at the outlet of the heat exchanger is controlled via the volumetric flow rate of the flue gas flowing through the heat exchanger.

8. A process as claimed in claim 1, wherein an exhaust gas is used which originates from the heterogeneously catalyzed gas-phase oxidation of n-butane and/or n-butenes to maleic anhydride, of o-xylene to phthalic anhydride, of propene to acrylic acid, of isobutene to methacrylic acid, of 1,2-ethanediol to glyoxal and/or of ethene to ethylene oxide
